(19) United States
(12) Patent Application Publication
(10) Pub. No.: US 2014/0296106 A1
(43) Pub. Date: Oct. 2, 2014

(54) METHODS FOR SCREENING SUBSTANCES CAPABLE OF MODULATING THE REPLICATION OF AN INFLUENZA VIRUS

(71) Applicant: INSERM (INSTITUT NATIONAL DE LA SANTÉ ET DE LA RECHERCHE MÉDICALE), Paris (FR)

(72) Inventors: Vincent Lotteau, Lyon (FR); Benoit De Chassey, Lyon (FR); Patrice Andre, Lyon (FR); Chantal Rabourdin-Combe, Lyon (FR); Lionel Tafforeau, Namur (BE); Thibault Chantier, Lyon (FR); Anne Aublin-Gex, Lyon (FR)

(73) Assignee: INSERM (INSTITUT NATIONAL DE LA SANTE ET DE LA RECHERCHE MEDICALE), Paris (FR)

(21) Appl. No.: 14/117,955

(22) PCT Filed: May 16, 2012
(86) PCT No.: PCT/EP2012/059189

§ 371 Date: Jun. 17, 2014

(30) Foreign Application Priority Data
May 16, 2011 (EP) 11305587.5

(51) Int. Cl. G01N 33/50

(52) U.S. Cl. CPC G01N 33/5023
USPC 506/10; 435/5

(57) ABSTRACT

The present invention relates to methods for screening substances capable of modulating the replication of an influenza virus. More particularly, the present invention relates to methods for screening a plurality of substances capable of modulating the replication of an influenza virus in a host cell comprising the step consisting of identifying a substance that modulates the specific interaction of a host cell protein with a viral protein required for viral replication as depicted in table 1 or identifying a substance that modulates the specific interaction of a first host cell protein as depicted in table 1 with a second host cell protein present in cellular network of the first host cell protein or identifying a substance that modulates the expression of a host cell protein as depicted in table 1, or identifying a substance that modulates the activity of a host cell protein as depicted in table 1

### FIELD OF THE INVENTION

The present invention relates to methods for screening substances capable of modulating the replication of an influenza virus.

### BACKGROUND OF THE INVENTION

Influenza viruses are one of the most ubiquitous viruses present in the world, affecting both humans and livestock. Influenza results in an economic burden, morbidity and even mortality, which are significant.

The influenza virus is an RNA enveloped virus with a particle size of about 125 nm in diameter. It consists basically of an internal nucleocapsid or core of ribonucleic acid (RNA) associated with nucleoprotein, surrounded by a viral envelope with a lipid bilayer structure and external glycoproteins. The inner layer of the viral envelope is composed predominantly of matrix proteins and the outer layer mostly of host-derived lipid material. Influenza virus comprises two surface antigens, glycoproteins neuraminidase (NA) and haemagglutinin (HA), which appear as spikes, 10 to 12 nm long, at the surface of the particles. It is these surface proteins, particularly the haemagglutinin that determine the antigenic specificity of the influenza subtypes. Virus strains are classified according to host species of origin, geographic site and year of isolation, serial number, and, for influenza A, by serological properties of subtypes of HA and NA. 16 HA subtypes (HI-HI6) and nine NA subtypes (N1-1-N9) have been identified for influenza A viruses. Viruses of all HA and NA subtypes have been recovered from aquatic birds, but only three HA subtypes (HI, H2, and H3) and two NA subtypes (N1 and N2) have established stable lineages in the human population since 1918. Only one subtype of HA and one of NA are recognised for influenza B viruses.

The genome of the virus consists of 8 segments and encodes 10 polypeptides. Replication and transcription of influenza virus RNA requires four virus encoded proteins: the NP and the three components of the viral RNA-dependent RNA polymerase, PB1, PB2 and PA. For example, the NP is the major structural component of the virion which interacts with genomic RNA, and is required for antitermination during RNA synthesis. NP is also required for elongation of RNA chains but not for initiation. Another example is NS1. NS1 is a major non-structural protein expressed by influenza A viruses in infected cells, whose role in infection is not clear. Studies of viruses carrying temperature-sensitive NS1 alleles point to a regulatory role for NS1 in viral gene-expression and/or replication, which is also consistent with its preferentially nuclear accumulation. Its expression has been shown to interfere with cellular functions in a variety of ways. These effects have been suggested to be mediated through NS1's observed interactions with a variety of RNA's, including single- and double-stranded influenza vRNA, poly-adenosine RNA, and spliceosomal U6 RNA. Despite these studies involving the interaction of NS1 with various RNAs, no host proteins that interact with NS1 during infection have previously been identified or characterized.

Little is known about host cell functions which contribute to the intracellular replication of influenza viruses, and cellular factors have not been characterized which directly interact with the viral proteins, much less cellular factor/viral interactions that can for example be used as targets for therapeutic intervention.

### SUMMARY OF THE INVENTION

The present invention relates to methods for screening a plurality of substances capable of modulating the replication of an influenza virus in a host cell comprising the step consisting of
identifying a substance that modulates the specific interaction of a host cell protein with a viral protein required for viral replication as depicted in table 1 or
identifying a substance that modulates the specific interaction of a first host cell protein as depicted in table 1 with a second host cell protein present in cellular network of the first host cell protein or
identifying a substance that modulates the expression of a host cell protein as depicted in table 1, or
identifying a substance that modulates the activity of a host cell protein as depicted in table 1

### DETAILED DESCRIPTION OF THE INVENTION

The invention is based, in part, on the inventors' discovery of novel interactions between influenza viral proteins and human host cell proteins. Said interactions are reported in the table 1. The host cell proteins represent accessory proteins required for replication of influenza virus. A viral infection can indeed be described as a network of interaction between viral and host proteins. On one hand, viruses hijack the function of the host proteins for their own replication. On the other hand, the cells respond to the infection with antiviral defence measures accomplished by host proteins. Hence, influenza cellular interactors can be used to drive the discovery of new potential influenza replication modulators. A modulator of replication is a substance able to enhance or inhibit viral replication. As cellular interactors play a central role in the infectious process, such a substance is typically i) a molecule that modulates the interaction between the cellular interactor and the viral protein, or ii) a molecule that modulates the interaction between the cellular interactor and any cellular protein or iii) a molecule that modulates a molecular function of the cellular interactor (eg an enzymatic function).

Accordingly, the present invention relates to the identification of host cell proteins that interact with viral proteins required for virus replication, and high throughput assays to identify substances that interfere with the specific interaction between the viral and host cell protein. Interfering substances that inhibit viral replication can be used therapeutically to treat viral infection. Alternatively, interfering substances that enhance viral replication can be used the production of influenza virus vaccines.

All the human and influenza genes and proteins such as defined in the table 1 are known per se by the skilled man in the art.

The present invention describes various biological assays that may be used for screening substances that can modulate the influenza virus replication in a host cell.

The present invention also relates to methods for screening a plurality of substances capable of modulating the replication of an influenza virus in a host cell comprising the step consisting of
identifying a substance that modulates the specific interaction of a host cell protein with a viral protein required for viral replication as depicted in table 1 or
identifying a substance that modulates the specific interaction of a first host cell protein as depicted in table 1 with a second host cell protein present in cellular network of the first host cell protein or
identifying a substance that modulates the expression of a host cell protein as depicted in table 1, or
identifying a substance that modulates the activity of a host cell protein as depicted in table 1

Biological Assays for Identifying a Substance that Modulates the Specific Interaction of a Host Cell Protein with a Viral Protein Required for Viral Replication:

A particular aspect of the invention relates to an assay for identifying a substance that modulates the specific interaction of a host cell protein with a viral protein required for viral replication as depicted in table 1 comprising:

(a) contacting a protein or peptide containing an amino acid sequence corresponding to the binding site of the host cell protein with a protein or peptide having an amino acid sequence corresponding to the binding site of the viral protein, under conditions and for a time sufficient to permit binding and the formation of a complex, in the presence of a test substance, and

(b) detecting the formation of a complex, in which the ability of the test substance to modulates the interaction between the host cell protein and the viral protein is indicated by a decrease in complex formation as compared to the amount of complex formed in the absence of the test substance.

Accordingly, the assay as above described may be useful for identifying molecules that inhibit or enhance the specific interaction of the host cell protein with the viral protein.

In one embodiment the step b) consists in generating physical values which illustrate or not the ability of said test substance to modulates the interaction between said influenza virus protein and said host cell protein and comparing said values with standard physical values obtained in the same assay performed in the absence of the said test substance. The “physical values” that are referred to above may be of various kinds depending of the binding assay that is performed, but notably encompass light absorbance values, radioactive signals and intensity value of fluorescence signal. If after the comparison of the physical values with the standard physical values, it is determined that the said test substance modulatess the binding between said influenza virus protein and said host cell protein, then the candidate is positively selected at step b).

The substances that modulates the interaction between the influenza virus protein and host cell protein encompass those substances that bind either to influenza virus protein or to host cell protein, provided that the binding of the said substances of interest then prevents the interaction between influenza virus protein and host cell protein.

In one embodiment, any protein or peptide of the invention is labelled with a detectable molecule.

According to the invention, said detectable molecule may consist of any substance or substance that is detectable by spectroscopic, photochemical, biochemical, immunochemical or chemical means. For example, useful detectable molecules include radioactive substance (including those comprising ³²P, ²⁵S, ³H, or ¹²⁵I), fluorescent dyes (including 5-bromodesosyrudin, fluorescein, acetylaminofluorene or digoxigenin), fluorescent proteins (including GFPs and YFPs), or detectable proteins or peptides (including biotin, polyhistidine tails or other antigen tags like the HA antigen, the FLAG antigen, the c-myc antigen and the DNP antigen).

According to the invention, the detectable molecule is located at, or bound to, an amino acid residue located outside the said amino acid sequence of interest, in order to minimise or prevent any artifact for the binding between said polypeptides or between the test substance and or any of said polypeptides.

In another particular embodiment, the polypeptides of the invention are fused with a GST tag (Glutathione S-transferase). In this embodiment, the GST moiety of the said fusion protein may be used as detectable molecule. In the said fusion protein, the GST may be located either at the N-terminal end or at the C-terminal end. The GST detectable molecule may be detected when it is subsequently brought into contact with an anti-GST antibody, including with a labelled anti-GST antibody. Anti-GST antibodies labelled with various detectable molecules are easily commercially available.

In another particular embodiment, proteins of the invention are fused with a poly-histidine tag. Said poly-histidine tag usually comprises at least four consecutive hisitidine residues and generally at least six consecutive histidine residues. Such a polypeptide tag may also comprise up to 20 consecutive histidine residues. Said poly-histidine tag may be located either at the N-terminal end or at the C-terminal end In this embodiment, the poly-histidine tag may be detected when it is subsequently brought into contact with an anti-poly-histidine antibody, including with a labelled anti-poly-histidine antibody. Anti-poly-histidine antibodies labelled with various detectable molecules are easily commercially available.

In a further embodiment, the proteins of the invention are fused with a protein moiety consisting of either the DNA binding domain or the activator domain of a transcription factor. Said protein moiety domain of transcription may be located either at the N-terminal end or at the C-terminal end. Such a DNA binding domain may consist of the well-known DNA binding domain of LexA protein originating form E. Coli. Moreover said activator domain of a transcription factor may consist of the activator domain of the well-known Ga14 protein originating from yeast.

In one embodiment of the assay according to the invention, the proteins of the invention comprise a portion of a transcription factor. In said assay, the binding together of the first and second portions generates a functional transcription factor that binds to a specific regulatory DNA sequence, which in turn induces expression of a reporter DNA sequence, said expression being further detected and/or measured. A positive detection of the expression of said reporter DNA sequence means that an active transcription factor is formed, due to the binding together of said first influenza virus protein and second host cell protein.

Usually, in a two-hybrid assay, the first and second portion of a transcription factor consist respectively of (i) the DNA binding domain of a transcription factor and (ii) the activator domain of a transcription factor. In some embodiments, the DNA binding domain and the activator domain both originate from the same naturally occurring transcription factor. In some embodiments, the DNA binding domain and the activator domain originate from distinct naturally occurring factors, while, when bound together, these two portions form an active transcription factor. The term “portion” when used herein for transcription factor, encompass complete proteins involved in multi protein transcription factors, as well as specific functional protein domains of a complete transcription factor protein.

Therefore in one embodiment of the invention, the assay of the invention comprises the following steps:

(1) providing a host cell expressing:
a first fusion polypeptide between (i) a influenza virus protein as defined above and (ii) a first protein portion of transcription factor
a second fusion polypeptide between (i) a host cell protein as defined above and (ii) a second portion of a transcription factor

said transcription factor being active on DNA target regulatory sequence when the first and second protein portion are bound together and

said host cell also containing a nucleic acid comprising (i) a regulatory DNA sequence that may be activated by said active transcription factor and (ii) a DNA report sequence that is operatively linked to said regulatory sequence

(2) bringing said host cell provided at step 1) into contact with a test substance to be tested

(3) determining the expression level of said DNA reporter sequence

The expression level of said DNA reporter sequence that is determined at step (3) above is compared with the expression of said DNA reporter sequence when step (2) is omitted. A different expression level of said DNA reporter sequence in the presence of the test substance means that the said test substance effectively modulatess the binding between influenza virus protein and host cell protein and that said test substance may be positively selected.

Suitable host cells include, without limitation, prokaryotic cells (such as bacteria) and eukaryotic cells (such as yeast cells, mammalian cells, insect cells, plant cells, etc.). However preferred host cell are yeast cells and more preferably a Saccharomyces cerevisiae cell or a Schizosaccharomyces pombe cell.

Similar systems of two-hybrid assays are well known in the art and therefore can be used to perform the assay according to the invention (see. Fields et al. 1989; Vasavada et al. 1991; Fearon et al. 1992; Dang et al., 1991, Chien et al. 1991, U.S. Pat. No. 5,283,173, U.S. Pat. No. 5,667,973, U.S. Pat. No. 5,468,614, U.S. Pat. No. 5,525,490 and U.S. Pat. No. 5,637,463). For instance, as described in these documents, the Ga14 activator domain can be used for performing the assay according to the invention. Ga14 consists of two physically discrete modular domains, one acting as the DNA binding domain, the other one functioning as the transcription-activation domain. The yeast expression system described in the foregoing documents takes advantage of this property. The expression of a Ga11-LacZ reporter gene under the control of a Ga14-activated promoter depends on the reconstitution of Ga14 activity via protein-protein interaction. Colonies containing interacting polypeptides are detected with a chromogenic substrate for β-galactosidase. A compete kit (MATCHMAKER™) for identifying protein-protein interactions is commercially available from Clontech.

So in one embodiment, a first influenza virus protein as above defined is fused to the DNA binding domain of Ga14 and the second host cell protein as above defined is fused to the activation domain of Ga14.

The expression of said detectable marker gene may be assessed by quantifying the amount of the corresponding specific mRNA produced. However, usually the detectable marker gene sequence encodes for detectable protein, so that the expression level of the said detectable marker gene is assessed by quantifying the amount of the corresponding protein produced. Techniques for quantifying the amount of mRNA or protein are well known in the art. For example, the detectable marker gene placed under the control of regulatory sequence may consist of the β-galactosidase as above described.

In another one embodiment, the assay according to the invention comprises a step of subjecting to a gel migration assay the mixture of the first influenza virus protein and the second host cell protein as above defined, with or without the test substance to be tested and then measuring the binding of the said polypeptides altogether by performing a detection of the complexes formed between said polypeptides. The gel migration assay can be carried out as known by the one skilled in the art.

Therefore in one embodiment of the invention, the assay of the invention comprises the following steps:

(1) providing a first influenza virus protein and a second host cell protein as defined above

(2) bringing into contact the test substance to be tested with said polypeptides

(3) performing a gel migration assay a suitable migration substrate with said polypeptides and said test substance as obtained at step (2)

(4) detecting and quantifying the complexes formed between said polypeptides on the migration assay as performed at step (3).

The presence or the amount of the complexes formed between the proteins are then compared with the results obtained when the assay is performed in the absence of the test substance to be tested. Therefore, when no complexes between the proteins is detected or, alternatively when those complexes are present in a lower amount compared to the amount obtained in the absence of the test substance, means that the test substance may be selected as an inhibitor of the specific interaction between said host protein and said viral protein.

The detection of the complexes formed between the said two proteins may be easily performed by staining the migration gel with a suitable dye and then determining the protein bands corresponding to the protein analysed since the complexes formed between the first and the second proteins possess a specific apparent molecular weight. Staining of proteins in gels may be done using the standard Coomassie brilliant blue (or PAGE blue), Amido Black, or silver stain reagents of different kinds. Suitable gels are well known in the art such as sodium dodecyl (lauryl) sulfate-polyacrylamide gel. In a general manner, western blotting assays are well known in the art and have been widely described (Rybicki et al., 1982; Towbin et al. 1979; Kurien et al. 2006).

In a particular embodiment, the protein bands corresponding to the proteins submitted to the gel migration assay can be detected by specific antibodies. It may used both antibodies directed against the influenza virus proteins and antibodies specifically directed against the host cell proteins.

In another embodiment, the said two proteins are labelled with a detectable antigen as above described. Therefore, the proteins bands can be detected by specific antibodies directed against said detectable antigen. Preferably, the detectable antigen conjugates to the influenza virus protein is different from the antigen conjugated to the host cell protein. For instance, the first influenza virus protein can be fused to a GST detectable antigen and the second host cell protein can be fused with the HA antigen. Then the protein complexes formed between the two proteins may be quantified and determined with antibodies directed against the GST and HA antigens respectively.

In another embodiment, the assay of the present invention includes the use of an optical biosensor such as described by Edwards et al. (1997) or also by Szabo et al. (1995). This technique allows the detection of interactions between molecules in real time, without the need of labelled molecules. This technique is indeed bases on the surface plasmon resonance (SPR) phenomenon. Briefly, a first protein partner is attached to a surface (such as a carboxymethyl dextran matrix). Then the second protein partner is incubated with the previously immobilised first partner, in the presence or absence of the test substance to be tested. Then the binding including the binding level or the absence of binding between said protein partners is detected. For this purpose, a light beam is directed towards the side of the surface area of the substrate that does not contain the sample to be tested and is reflected by said surface. The SPR phenomenon causes a decrease in the intensity of the reflected light with a combination of angle and wavelength. The binding of the first and second protein partner causes a change in the refraction index on the substrate surface, which change is detected as a change in the SPR signal.

In another one embodiment of the invention, the assay includes the use of affinity chromatography.

Test substances for use in the assay above can also be selected by any immunoaffinity chromatography technique using any chromatographic substrate onto which (i) the first influenza virus protein or (ii) the second host cell protein as above defined, has previously been immobilised, according to techniques well known from the one skilled in the art. Briefly, the influenza virus protein or the host cell protein as above defined, may be attached to a column using conventional techniques including chemical coupling to a suitable column matrix such as agarose, Affi Gel®, or other matrices familiar to those of skill in the art. In some embodiment of this method, the affinity column contains chimeric proteins in which the influenza virus protein or host cell protein as above defined, is fused to glutathion-s-transferase (GST). Then a test substance is brought into contact with the chromatographic substrate of the affinity column previously, simultaneously or subsequently to the other protein among the said first and second protein. The after washing, the chromatography substrate is eluted and the collected elution liquid is analysed by detection and/or quantification of the said later applied first or second protein, so as to determine if, and/or to which extent, the test substance has impaired or not the binding between (i) first influenza virus protein and (ii) the second host cell protein.

In another one embodiment of the assay according to the invention, the first influenza virus protein and the second host cell protein as above defined are labelled with a fluorescent molecule or substrate. Therefore, the potential alteration effect of the test substance to be tested on the binding between the first influenza virus protein and the second host cell protein as above defined is determined by fluorescence quantification.

For example, the first influenza virus protein and the second host cell protein as above defined may be fused with auto-fluorescent polypeptides, as GFP or YFPs as above described. The first influenza virus protein and the second host cell protein as above defined may also be labelled with fluorescent molecules that are suitable for performing fluorescence detection and/or quantification for the binding between said proteins using fluorescence energy transfer (FRET) assay. The first influenza virus protein and the second host cell protein as above defined may be directly labelled with fluorescent molecules, by covalent chemical linkage with the fluorescent molecule as GFP or YFP. The first influenza virus protein and the second host cell protein as above defined may also be indirectly labelled with fluorescent molecules, for example, by non covalent linkage between said polypeptides and said fluorescent molecule. Actually, said first influenza virus protein and second host cell protein as above defined may be fused with a receptor or ligand and said fluorescent molecule may be fused with the corresponding ligand or receptor, so that the fluorecent molecule can non-covalently bind to said first influenza virus protein and second host cell protein. A suitable receptor/ligand couple may be the biotin/streptavifin paired member or may be selected among an antigen/antibody paired member. For example, a protein according to the invention may be fused to a poly-histidine tail and the fluorescent molecule may be fused with an antibody directed against the poly-histidine tail.

As already specified, the assay according to the invention encompasses determination of the ability of the test substance to modulates the interaction between the influenza virus protein and the host cell protein as above defined by fluorescence assays using FRET. Thus, in a particular embodiment, the first influenza virus protein as above defined is labelled with a first fluorophore substance and the second host cell protein is labelled with a second fluorophore substance. The first fluorophore substance may have a wavelength value that is substantially equal to the excitation wavelength value of the second fluorophore, whereby the bind of said first and second proteins is detected by measuring the fluorescence signal intensity emitted at the emission wavelength of the second fluorophore substance. Alternatively, the second fluorophore substance may also have an emission wavelength value of the first fluorophore, whereby the binding of said and second proteins is detected by measuring the fluorescence signal intensity emitted at the wavelength of the first fluorophore substance.

The fluorophores used may be of various suitable kinds, such as the well-known lanthanide chelates. These chelates have been described as having chemical stability, long-lived fluorescence (greater than 0.1 ms lifetime) after bioconjugation and significant energy-transfer in specificity bioaffinity assay. Document U.S. Pat. No. 5,162,508 discloses bipyridine cryptates. Polycarboxylate chelators with TEKES type photosensitizers (EP0203047A1) and terpyridine type photosensitizers (EP0649020A1) are known. Document WO96/00901 discloses diethylenetriaminepentaacetic acid (DPTA) chelates which used carbostyril as sensitizer. Additional DPT chelates with other sensitizer and other tracer metal are known for diagnostic or imaging uses (e.g., EP0450742A1).

In a preferred embodiment, the fluorescence assay consists of a Homogeneous Time Resolved Fluorescence (HTRF) assay, such as described in document WO 00/01663 or U.S. Pat. No. 6,740,756, the entire content of both documents being herein incorporated by reference.

HTRF is a TR-FRET based technology that uses the principles of both TRF (time-resolved fluorescence) and FRET. More specifically, the one skilled in the art may use a HTRF assay based on the time-resolved amplified cryptate emission (TRACE) technology as described in Leblanc et al. (2002). The HTRF donor fluorophore is Europium Cryptate, which has the long-lived emissions of lanthanides coupled with the stability of cryptate encapsulation. XL665, a modified allophycocyanin purified from red algae, is the HTRF primary acceptor fluorophore. When these two fluorophores are brought together by a biomolecular interaction, a portion of the energy captured by the Cryptate during excitation is released through fluorescence emission at 620 nm, while the remaining energy is transferred to XL665. This energy is then released by XL665 as specific fluorescence at 665 nm. Light at 665 nm is emitted only through FRET with Europium. Because Europium Cryptate is always present in the assay, light at 620 nm is detected even when the biomolecular interaction does not bring XL665 within close proximity.

Therefore in one embodiment, the assay may therefore comprises the steps of:

(1) bringing into contact a pre-assay sample comprising:
a first influenza virus protein fused to a first antigen,
a second host cell protein fused to a second antigen,
a test substance to be tested

(2) adding to the said pre assay sample of step (1):

at least one antibody labelled with a European Cryptate which is specifically directed against the first said antigen,

at least one antibody labelled with XL665 directed against the second said antigen,

(3) illuminating the assay sample of step (2) at the excitation wavelength of the said European Cryptate,
(4) detecting and/or quantifying the fluorescence signal emitted at the XL665 emission wavelength.

(5) comparing the fluorescence signal obtained at step (4) to the fluorescence obtained wherein pre assay sample of step (1) is prepared in the absence of the test substance to be tested.

If at step (5) as above described, the intensity value of the fluorescence signal is lower than the intensity value of the fluorescence signal found when pre assay sample of step (1) is prepared in the absence of the test substance to be tested, then the test substance may be selected as an inhibitor of the specific interaction between said host protein and said viral protein.

Antibodies labelled with a European Cryptate or labelled with XL665 can be directed against different antigens of interest including GST, poly-histidine tail, DNP, c-myx, HA antigen and FLAG which include. Such antibodies encompass those which are commercially available from CisBio (Bedfors, Mass., USA), and notably those referred to as 61GSTKLA or 61HISKLB respectively.

Alternatively, in another one embodiment of the assay according to the invention, the modulation of the specific interaction between the host cell protein and the viral protein may be determined using isothermal titration calorimetry (ITC). Typically, Isothermal titration calorimetry (ITC) experiments were performed with a ITC titration calorimeter (suc as provide by Microcal Inc., Northampton, Mass., USA). Solutions comprising host cell protein (or alternatively viral protein) are then prepared. The enthalpy change resulting from the contacting with the viral protein (or alternatively host cell protein) was obtained through integration of the calorimetric signal. Different ITC experimental formats are typically employed in order to obtain compound dissociation constants (Kd's) over a wide range of affinities.

Assays for Identifying a Substance that Modulates the Specific Interaction of a Host Cell Protein as Depicted in Table 1 and a Second Host Cell Protein Present in Cellular Network of Said First Host Cell Protein:

Another particular aspect of the invention relates to an assay for identifying a substance that modulates the specific interaction of a first host cell protein as depicted in table 1 with a second host cell protein present in cellular network of the first host cell protein comprising:

(a) contacting a protein or peptide containing an amino acid sequence corresponding to the binding site of the first host cell protein with a protein or peptide having an amino acid sequence corresponding to the binding site of the second host cell protein, under conditions and for a time sufficient to permit binding and the formation of a complex, in the presence of a test substance, and

(b) detecting the formation of a complex, in which the ability of the test substance to modulates the interaction between the first host cell protein and the second host cell protein is indicated by a decrease in complex formation as compared to the amount of complex formed in the absence of the test substance.

Identification of the second host cell protein may be performed by gathering information from the prior art that described interactions between two host cell protein. Alternatively, the second host cell protein may be determined by performing a two hybrid assay.

All the methods as described for the above described assays may be used.

Assays for Identifying a Substance that Modulate the Expression of a Host Cell Protein as Depicted in Table 1:

Another particular aspect of the invention relates to an assay for identifying a substance that modulates the expression of a host cell protein as depicted in table 1 comprising

determining whether the test substance modulates the expression of said host cell protein

In one embodiment, the assay comprises the steps of i) contacting the test with a cell transfected with a reporter gene operatively linked to all or part of the promoter of the gene encoding for the host cell protein, ii) assessing the level of expression of said reporter gene, and iii) identifying the test substance which modulates the expression of said reporter gene.

Abroad variety of host-expression vector systems may be utilized to express the genes used in the assay. These include, but are not limited to, mammalian cell systems such as human cell lines derived from colon adenocarcinoma including HT-29, Caco-2, SW480, HTC116, The mammalian cell systems may harbour recombinant expression constructs containing promoters derived from the genome of mammalian cells or from mammalian viruses (e.g., the adenovirus late promoter or the vaccine virus 7.5K promoter).

Additional host-expression vector systems include, but are not limited to, microorganisms such as bacteria (e.g., E. 5 coli or B. subtilis) transformed with recombinant bacteriophage DNA, plasmid DNA, or cosmid DNA expression vectors containing PTK or adaptor protein coding sequences; yeast (e.g., Saccharomyces, Pichia) transformed with recombinant yeast expression vectors containing the protein or peptide oding sequences; insect cell systems, such as Sf9 or Sf21 infected with recombinant virus expression vectors (e.g., baculovirus) containing the protein or peptide coding sequences; amphibian cells, such as Xenopus oocytes; or plant cell systems infected with recombinant virus express-15 sion vectors (e.g., cauliflower mosaic virus, CaMV; tobacco mosaic virus, TMV) or transformed with recombinant plamid expression vectors (e.g., Ti plasmid) containing the protein or peptide coding sequence. Culture conditions for each of these cell types is specific and is known to those familiar with the art.

DNA encoding proteins to be assayed can be transiently or stably expressed in the cell lines by several methods known in the art, such as, calcium phosphate-mediated, DEAE-dextran mediated, liposomal-mediated, viral-mediated, electroporation-mediated and microinjection delivery. Each of these methods may require optimization of assorted experimental parameters depending on the DNA, cell line, and the type of assay to be subsequently employed.

In addition native cell lines that naturally carry and express the nucleic acid sequences for the target protein may be used.

In a particular embodiment, the invention is directed to a method, which comprises the steps of i) contacting the test substance with a cell capable of expressing the gene encoding for the host cell protein, ii) assessing the level of expression of said gene, and iii) identifying the test substance which modulates the expression of said gene. In one embodiment, the level of expression is assessed by determining the level of transcription of said gene. In a further embodiment, the determination of the level of translation of said gene is effected by means of an immunoassay.

Determination of the expression level of a gene can be performed by a variety of techniques. Generally, the expression level as determined is a relative expression level.

More preferably, the determination comprises contacting the sample with selective reagents such as probes, primers or ligands, and thereby detecting the presence, or measuring the amount, of polypeptide or nucleic acids of interest originally in the sample. Contacting may be performed in any suitable device, such as a plate, microtiter dish, test tube, well, glass, column, and so forth In specific embodiments, the contacting is performed on a substrate coated with the reagent, such as a nucleic acid array or a specific ligand array. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as a nucleic acid hybrid or an antibody-antigen complex, to be formed between the reagent and the nucleic acids or polypeptides of the sample.

In a preferred embodiment, the expression level may be determined by determining the quantity of mRNA.

Methods for determining the quantity of mRNA are well known in the art. For example the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Preferably quantitative or semi-quantitative RT-PCR is preferred. Real-time quantitative or semi-quantitative RT-PCR is particularly advantageous.

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e.g. avidin/biotin).

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210)

Other methods for determining the expression level of said genes include the determination of the quantity of proteins encoded by said genes.

Such methods comprise contacting a biological sample with a binding partner capable of selectively interacting with a marker protein present in the sample. The binding partner is generally an antibody that may be polyclonal or monoclonal, preferably monoclonal.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with an antibody against the protein to be tested. A biological sample containing or suspected of containing the marker protein is then added to the coated wells. After a period of incubation sufficient to allow the formation of antibody-antigen complexes, the plate (s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

Assays for Identifying a Substance that Modulate the Activity of a Host Cell Protein as Depicted in Table 1:

Another particular aspect of the invention relates to an assay for identifying a substance that modulates the activity of a host cell protein as depicted in table 1 comprising:

(a) contacting the host cell protein with a test substance, and

(b) determining whether the test substance modulates the activity of said host cell protein

The activity of a host cell protein may be easily determined by the skilled man in the art. For example, for enzymes, various enzymatic assay may be used for determined whether the test substance could modulate the activity of said host cell protein. Other functional assays may be used and may be determined by the information disclosed in the prior art.

Test Substances:

The test substance of the invention may be selected from a library of substances previously synthesised, or a library of substances for which the structure is determined in a database, or from a library of substances that have been synthesised de novo. The test substance may be selected from the group of (a) proteins or peptides, (b) nucleic acids and (c) organic or chemical substances. Illustratively, libraries of pre-selected candidate nucleic acids may be obtained by performing the SELEX method as described in documents U.S. Pat. No. 5,475,096 and U.S. Pat. No. 5,270,163. Further illustratively, the test substance may be selected from the group of antibodies directed against said influenza virus protein and said host cell proteins as above described.

Methods for Screening Substances for Inhibiting the Replication Capacity of an Influenza Virus Replication:

A particular aspect of the invention relates to method for screening substances useful for inhibiting the replication capacity of an influenza virus in a host cell comprising the steps consisting of (a) selecting a test substance by performing at least one assay as described above (b) determining whether said substance inhibits the replication capacity of said influenza virus in a host cell and (c) positively selecting the test substance capable of inhibiting the replication capacity of said influenza virus in said host cell.

In one embodiment, the method comprises the steps consisting of i) infecting said host cell with said influenza virus and ii) culturing said infected cell in presence of the test substance, iii) comparing the replicating capacity of the virus with the replication capacity determined in the absence of the test substance and iv) positively selecting the test substance that provides a decrease in the replication capacity of the virus.

The term “decrease in the replication capacity,” as used herein with reference to a viral phenotype, means that the virus grows to a lower titer in the presence of a substance as above described relative to the virus grown in the absence of said substance. In one embodiment, the presence of said substance which will inhibit the ability of an influenza virus to replicate in a host cell by at least about 10%, or by at least about 20%, or by at least about 30%, or by at least about 40%, or by at least about 50%, or by at least about 60%, or by at least about 70%, or by at least about 80%, or by at least about 90%, or by at least about 100%, or by at least about 200%, or by at least about 300%, or by at least about 400%, or by at least about 500% when compared to said influenza virus grown in the absence of said substance. Said replication capacity may be typically determined by quantifying viral particle formation as indicated by hemagglutinin (HA) titers measured in the supernatants of infected host cells, or by determining plaque forming unils (PFU) or neuramidase acivity.

Methods for Screening Substances for Increasing Capacity of an Influenza Virus Replication:

A particular aspect of the invention relates to method for screening substances useful for increasing the replication capacity of an influenza virus in a host cell comprising the steps consisting of (a) selecting a test substance by performing at least one assay as described above (b) determining whether said substance inhibits the replication of said influenza virus in a host cell and (c) positively selecting the test substancs capable of inhibiting the replication of said influenza virus in said host cell.

In one embodiment, the method comprises the steps consisting of i) infecting said host cell with said influenza virus and ii) culturing said infected cell in presence of the test substance, iii) comparing the replicating capacity of the virus with the replication capacity determined in the absence of the test substance and iv) positively selecting the test substances that provides an increase in the replication capacity of the virus.

The term “increase in the replication capacity,” as used herein with reference to a viral phenotype, means that the virus grows to a higher titer in the presence of a substance as above described relative to the virus grown in the absence of said substance. In one embodiment, the presence of said substance which will increase the ability of an influenza virus to replicate in a host cell by at least about 10%, or by at least about 20%, or by at least about 30%, or by at least about 40%, or by at least about 50%, or by at least about 60%, or by at least about 70%, or by at least about 80%, or by at least about 90%, or by at least about 100%, or by at least about 200%, or by at least about 300%, or by at least about 400%, or by at least about 500% when compared to said influenza virus grown in the absence of said substance. Said replication capacity may be typically determined by quantifying viral particle formation as indicated by hemagglutinin (HA) titers measured in the supernatants of infected host cells, or by determining plaque forming unils (PFU) or neuramidase acivity.

Influenza Virus Used in the Screening Methods:

According to the present invention, any influenza virus strain can be used. Preferably, said influenza virus strain corresponds to a clinical isolate of at least one circulating strain of an influenza A or B virus. For the production of a safe and effective vaccine it is indeed important that the selected influenza virus strains are closely related to the circulating strains. Type A viruses are principally classified into antigenic sub-types on the basis of two viral surface glycoproteins, hemagglutinin (HA) and neuraminidase (NA). There are currently 16 identified HA sub-types (designated H1 through H16) and 9 NA sub-types (N1 through N9) all of which can be found in wild aquatic birds. Of the 135 possible combinations of HA and NA, only four (H1N1, H1N2, H2N2, H5N1 and H3N2) have widely circulated in the human population since the virus was first isolated in 1933.

In a particular embodiment, the clinical isolate can be made into a high growth strain by reassortment with a high growth master donor strain, or by multiple passages of the clinical isolate in continuous mammalian cell lines, with selection of high growth variants. The clinical isolates are preferably reassorted with laboratory high growth master donor strains in culture, and the reassortants selected that have HA and NA genes from the isolates, and internal genes from the high growth master laboratory strains. For example, the resulting strain for the influenza A component can be a reassortant virus that contains internal genes from the master donor strain A/PR/8/34 (H1N1), which provides high growth in cells, as well as at least the HA gene coding for at least one surface antigen of the clinical isolate of the influenza virus (using known methods, e.g., according to Robertson et al., Biologicals 20:213-220 (1992)). Such reassortants can be made more rapidly than high growth strains made by multiple passages of the clinical isolates.

In a further preferred embodiment, the infection of the cells with influenza viruses is carried out at an m.o.i. (multiplicity of infection) of about 0.0001 to 10, preferably of 0.002 to 0.5.

Host Cells for Viral Replication:

According to the invention, any eukaryotic cell may be used in the screening method of the invention. Preferably said cell is a mammalian cell, but may be also an avian cell (e.g. egg). Typically said mammalian cells include but are not limited to cells from humans, dogs, cats, cattle, horses, sheep, pigs, goats, and rabbits. In a particular embodiment the cell is a human cell. In another particular embodiment said cell is a cell line. Non-limiting examples of cell lines that can be suitable for the invention include but are not limited to BS-C-1, CV-1, Vero, Vero 76, Vero C1008, Vero 76, Cos-1, Cos-7, FR11K-4, LLC-MK2 original, LLC-MK2 derivative, MDCK, RD, A549, MRC-5, KB, PER.C6, HEK-293 and CaCo-2 cells.

Typically, cells are cultured in a standard commercial culture medium, such as Dulbecco's modified Eagle's medium supplemented with serum (e.g., 10% fetal bovine serum), or in serum free medium, under controlled humidity and CO₂ concentration suitable for maintaining neutral buffered pH (e.g., at pH between 7.0 and 7.2). Suitable serum free media are described, for example, in U.S. Provisional Application No. 60/638,166, filed Dec. 23, 2004, and in U.S. Provisional Application No. 60/641,139, filed Jan. 5, 2005, each of which is hereby incorporated by reference in its entirety. Optionally, the medium contains antibiotics to prevent bacterial growth, e.g., penicillin, streptomycin, etc., and/or additional nutrients, such as L-glutamine, sodium pyruvate, nonessential amino acids, additional supplements to promote favorable growth characteristics, e.g., trypsin, (3-mercaptoethanol, and the like.

Cells for production of influenza virus can be cultured in serum-containing or serum free medium. In some case, e.g., for the preparation of purified viruses, it is desirable to grow the cells in serum free conditions. Cells can be cultured in small scale, e.g., less than 25 ml medium, culture tubes or flasks or in large flasks with agitation, in rotator bottles, oronmicrocarrierbeads (e.g., DEAE-Dextran microcarrier beads, such as Dormacell, Pfeifer & Langen; Superbead, Flow Laboratories; styrene copolymer-tri-methylamine beads, such as Hillex, SoloHill, Ann Arbor) in flasks, bottles or reactor cultures. Microcarrier beads are small spheres (in the range of 100-200 microns in diameter) that provide a large surface area for adherent cell growth per volume of cell culture. For example a single liter of medium can include more than 20 million microcarrier beads providing greater than 8000 square centimeters of growth surface. For commercial production of viruses, e.g., for vaccine production, it is often desirable to culture the cells in a bioreactor or fermenter. Bioreactors are available in volumes from under 1 liter to in excess of 100 liters, e.g., Cyto3 Bioreactor (Osmonics, Minnetonka, Minn.); NBS bioreactors (New Brunswick Scientific, Edison, N.J.); laboratory and commercial scale bioreactors from B. Braun Biotech International (B. Braun Biotech, Melsungen, Germany).

The cells can be grown in culture under conditions permissive for replication and assembly of viruses. In embodiments, cells can be cultured at a temperature below about 37° C., preferably at a temperature equal to, or less than, about 35° C. Typically, the cells are cultured at a temperature between about 32° C. and about 35° C. In some embodiments, the cells are cultured at a temperature between about 32° C. and 34° C., e.g., at about 33° C.

The culturing of the cells is carried out as a rule at a regulated pH which is preferably in the range from pH 6.6 to pH 7.8, in particular in the range from pH 6.8 to pH 7.3.

Furthermore, the pO2 value can advantageously be regulated and is then as a rule between 25% and 95%, in particular between 35% and 60% (based on the air saturation).

In a particular embodiment, a protease is added to the culture medium of the cells. The addition of the protease which brings about the cleavage of the precursor protein of hemagglutinin and thus the adsorption of the viruses on the cells, can be carried out according to the invention shortly before, simultaneously to or shortly after the infection of the cells with influenza viruses. If the addition is carried out simultaneously to the infection, the protease can either be added directly to the cell culture to be infected or, for example, as a concentrate together with the virus inoculate. The protease is preferably a serine protease, and particularly preferably trypsin. Typically, trypsin may be added to the cell culture to a final concentration of 1 to 200 μg/ml, preferably 5 to 50 μg/ml, and particularly preferably 5 to 30 μg/ml in the culture medium.

Methods for the Treatment or Prevention of Influenza Virus Infections:

As described above, the methods of the present invention are particularly useful for screening a plurality of substances that may be used for the treatment or prevention of influenza infections as described infra.

As used herein, the term “influenza infection” has its general meaning in the art and refers to the disease caused by an infection with an influenza virus. In some embodiments of the invention, influenza infection is associated with Influenza virus A or B. In some embodiments of the invention, influenza infection is associated with Influenza virus A. In some specific embodiments of the invention, influenza infection is cause by influenza virus A that is H1N1, H2N2, H3N₂ or H5N1.

The subject can be human or any other animal (e.g., birds and mammals) susceptible to influenza infection (e.g. domestic animals such as cats and dogs; livestock and farm animals such as horses, cows, pigs, chickens, etc.). Typically said subject is a mammal including a non-primate (e.g., a camel, donkey, zebra, cow, pig, horse, goat, sheep, cat, dog, rat, and mouse) and a primate (e.g., a monkey, chimpanzee, and a human). In certain embodiments, a subject is a non-human animal. In some embodiments, a subject is a farm animal or pet. In another embodiment, a subject is a human. In another embodiment, a subject is a human infant. In another embodiment, a subject is a human child. In another embodiment, a subject is a human adult. In another embodiment, a subject is an elderly human. In another embodiment, a subject is a premature human infant.

For example, therapeutic treatments includes the reduction or amelioration of the progression, severity and/or duration of influenza infections, or the amelioration of one or more symptoms (specifically, one or more discernible symptoms) of influenza infections, resulting from the administration of at least one substance selected by the above mentioned screening method. In specific embodiments, the therapeutic treatment includes the amelioration of at least one measurable physical parameter of an influenza infection. In other embodiments the therapeutic treatment includes the inhibition of the progression of an influenza infection, either physically by, e.g., stabilization of a discernible symptom, physiologically by, e.g., stabilization of a physical parameter, or both. In other embodiments the therapeutic treatment includes the reduction or stabilization of influenza infections. Antiviral drugs can be used in the community setting to treat people who already have influenza to reduce the severity of symptoms and reduce the number of days that they are sick.

In a particular embodiment, the substances selected by the above mentioned screening method may be used in a prophylactic treatment. The terms “prophylaxis” or “prophylactic use” and “prophylactic treatment” as used herein, refer to any medical or public health procedure whose purpose is to prevent, rather than treat or cure a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a subject with the disease.

As used herein, prophylactic use includes the use in situations in which an outbreak has been detected, to prevent contagion or spread of the infection in places where a lot of people that are at high risk of serious influenza complications live in close contact with each other (e.g. in a hospital ward, daycare center, prison, nursing home, etc). It also includes the use among populations who require protection from the influenza but who either do not get protection after vaccination (e.g. due to weak immune system), or when the vaccine is unavailable to them, or when they cannot get the vaccine because of side effects. It also includes use during the two weeks following vaccination, since during that time the vaccine is still ineffective. Prophylactic use may also include treating a person who is not ill with the influenza or not considered at high risk for complications, in order to reduce the chances of getting infected with the influenza and passing it on to a high-risk person in close contact with him (for instance, healthcare workers, nursing home workers, etc).

Typically, the substances selected by the above mentioned screening method are administered to the subject in an effective amount. As used herein, an “effective amount” refers to an amount sufficient to elicit the desired biological response. In the present invention the desired biological response is to inhibit the replication of influenza virus, to reduce the amount of influenza viruses or to reduce or ameliorate the severity, duration, progression, or onset of a influenza virus infection, prevent the advancement of an influenza viruses infection, prevent the recurrence, development, onset or progression of a symptom associated with an influenza virus infection, or enhance or improve the prophylactic or therapeutic effect(s) of another therapy used against influenza infections. The precise amount of compound administered to a subject will depend on the mode of administration, the type and severity of the infection and on the characteristics of the subject, such as general health, age, sex, body weight and tolerance to drugs. The skilled artisan will be able to determine appropriate dosages depending on these and other factors. When co-administered with other anti viral agents, e.g., when coadministered with an anti-influenza medication, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, an effective amount should be assumed. For example, compounds described herein can be administered to a subject in a dosage range from between approximately 0.01 to 100 mg/kg body weight/day for therapeutic or prophylactic treatment.

Generally, dosage regimens can be selected in accordance with a variety of factors including the disorder being treated and the severity of the disorder; the activity of the specific compound employed; the specific composition employed; the age, body weight, general health, sex and diet of the subject; the time of administration, route of administration, and rate of excretion of the specific compound employed; the renal and hepatic function of the subject; and the particular compound or salt thereof employed, the duration of the treatment; drugs used in combination or coincidental with the specific compound employed, and like factors well known in the medical arts. The skilled artisan can readily determine and prescribe the effective amount of the compounds described herein required to treat, to prevent, inhibit (fully or partially) or arrest the progress of the disease.

Dosages of the compounds described herein can range from between about 0.01 to about 100 mg/kg body weight/day, about 0.01 to about 50 mg/kg body weight/day, about 0.1 to about 50 mg/kg body weight/day, or about 1 to about 25 mg/kg body weight/day. It is understood that the total amount per day can be administered in a single dose or can be administered in multiple dosing, such as twice a day (e.g., every 12 hours), tree times a day (e.g., every 8 hours), or four times a day (e.g., every 6 hours).

For therapeutic treatment, the compounds described herein can be administered to a subject within, for example, 48 hours (or within 40 hours, or less than 2 days, or less than 1.5 days, or within 24 hours) of onset of symptoms (e.g., nasal congestion, sore throat, cough, aches, fatigue, headaches, and chills/sweats). The therapeutic treatment can last for any suitable duration, for example, for 5 days, 7 days, 10 days, 14 days, etc.

For prophylactic treatment during a community outbreak, the compounds described herein can be administered to a subject within, for example, 2 days of onset of symptoms in the index case, and can be continued for any suitable duration, for example, for 7 days, 10 days, 14 days, 20 days, 28 days, 35 days, 42 days, etc.

Various types of administration methods can be employed in the invention, and are described in detail below.

In a particular embodiment the substances selected by the above mentioned screening method are used in combination with an additional suitable therapeutic agent, for example, an antiviral agent or a vaccine. When “combination therapy” is employed, an effective amount can be achieved using a first amount of a substance selected by the above mentioned screening method and a second amount of an additional suitable therapeutic agent (e.g. an antiviral agent or vaccine).

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

Coadministration encompasses administration of the first and second amounts of the compounds of the coadministration in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order.

Specific examples that can be co-administered with a substance selected by the above mentioned screening method include neuraminidase inhibitors. Examples of neuraminidase inhibitors include oseltamivir, oseltamivir carboxylate (GS4071; see e.g. Eisenberg et al., Antimicrob Agents Chemother. (1997) 41:1949-52), zanamivir, peramivir (RWJ-27021; BXC-1812, BioCryst), 2,3-didehydro-2-deoxy-N-acetylneuraminic acid (DANA), 2-deoxy-2,3-dehydro-N-trifluoroacetylneuraminic acid (FANA), A-322278, and A-315675 (see U.S. Pat. No. 6,455,571 to Maring et al, and Kati et al., Antimicrob Agents Chemother. (2002) 46:1014-21).

Specific examples that can be co-administered with a substance selected by the above mentioned screening method include M2 inhibitors. Examples of M2 inhibitors include include aminoadamantane compounds such as amantadine (1-amino-adamantane), rimantadine (1-(1-aminoethyl)adamantane), spiro[cyclopropane-1,2′-adamantan]-2-amine, spiro[pyrrolidine-2,2′-adamantane], spiro[piperidine-2,2′-adamantane], 2-(2-adamantyl)piperidine, 3-(2-adamantyl)pyrrolidine, 2-(1-adamantyl) piperidine, 241-adamantyppyrrolidine, and 2-(ladamantyl)-2-methyl-pyrrolidine; and M2-specific monoclonal antibodies (see e.g. US 20050170334; and Zebedee and Lamb, J. Virol. (1988) 62:2762-72).

Specific examples that can be co-administered with a substance selected by the above mentioned screening method include RNA polymerase inhibitors. As used herein, the term RNA polymerase inhibitor refers to an antiviral agent that inhibits the polymerase, protease, and/or endonuclease activity of the viral RNA polymerase complex or one of its subunits (i.e. PB1, PB2 and PA). Exemplary RNA polymerase inhibitors include antiviral nucleoside analogs such as ribavirin, viramidine, 6-fluoro-3-hydroxy-2pyrazinecarboxamide (T-705), 2′-deoxy-2′-fluoroguanosine, pyrazofurin, 3-deazaguanine, carbodine (see e.g. Shannon et al., Antimicrob Agents Chemother. (1981) 20:769-76), and cyclopenenyl cytosine (see e.g. Shigeta et al., Antimicrob Agents Chemother. (1988) 32:906-11); and the endonuclease inhibitor flutimide (see e.g. Tomassini et al., Antimicrob Agents Chemother. (1996) 40:1189-93).

Specific examples that can be co-administered with a substance selected by the above mentioned screening method include influenza-specific interfering oligonucleotides Examples of influenza-specific interfering oligonucleotides include siRNAs (see e.g. Zhou et al., Antiviral Res. (2007) 76:186-93), antisense oligonucleotides, phosphorothioate oligonucleotides, ribozymes (see e.g. U.S. Pat. No. 6,258,585 to Draper), morpholino oligomers and peptide nucleic acids (see e.g. Schubert and Kurreck, Handb Exp Pharmacol. (2006) 173:261-87).

Specific examples that can be co-administered with a substance selected by the above mentioned screening method include interferons. An “interferon” or “IFN”, as used herein, is intended to include any molecule defined as such in the literature, comprising for example any types of IFNs (type I and type II) and in particular, IFN-alpha, IFN-beta, INF-omega and IFN-gamma. The term interferon, as used herein, is also intended to encompass salts, functional derivatives, variants, muteins, fused proteins, analogs and active fragments thereof. In a preferred embodiment the interferon is interferon-alpha. Interferon-alpha includes, but is not limited to, recombinant interferon-α2a (such as ROFERON® interferon available from Hoffman-LaRoche, Nutley, N.J.), interferon-α2b (such as Intron-A interferon available from Schering Corp., Kenilworth, N.J., USA), a consensus interferon, and a purified interferon-α product.

In some embodiments, the compounds described herein can be co-administered with an influenza vaccine. Influenza vaccines, of all kinds, are usually trivalent vaccines. They generally contain antigens derived from two influenza A virus strains and one influenza B strain. A standard 0.5 ml injectable dose in most cases contains 15 ug of hemagglutinin antigen component from each strain, as measured by single radial immunodiffusion (SRD). The influenza virus strains to be incorporated into influenza vaccine each season are determined by the World Health Organization in collaboration with national health authorities and vaccine manufacturers.

In some embodiments, a combination therapy comprises active immunization with an influenza antigenic polypeptide (e.g. influenza hemagglutinin and the matrix 2 ectodomain polypeptides) or passive immunization with one or more neutralizing antibodies directed to an influenza antigenic polypeptide (e.g. antibodies raised against the influenza hemagglutinin and the matrix 2 ectodomain polypeptides).

The substances selected by the above mentioned screening method can be formulated into pharmaceutical compositions that further comprise a pharmaceutically acceptable carrier, diluent, adjuvant or vehicle. In one embodiment, the present invention relates to a pharmaceutical composition comprising a substance selected by the above mentioned screening method described above, and a pharmaceutically acceptable carrier, diluent, adjuvant or vehicle. In one embodiment, the present invention is a pharmaceutical composition comprising an effective amount of a compound of the present invention or a pharmaceutically acceptable salt thereof and a pharmaceutically acceptable carrier, diluent, adjuvant or vehicle. Pharmaceutically acceptable carriers include, for example, pharmaceutical diluents, excipients or carriers suitably selected with respect to the intended form of administration, and consistent with conventional pharmaceutical practices.

A pharmaceutically acceptable carrier may contain inert ingredients which do not unduly inhibit the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

The pharmaceutically acceptable carrier, adjuvant, or vehicle, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, its use is contemplated to be within the scope of this invention.

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The compositions described herein may be administered orally, parenterally, by inhalation spray, topically, rectally, nasally, buccally, vaginally or via an implanted reservoir depending on the severity of the infection being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Specifically, the compositions are administered orally, intraperitoneally or intravenously.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a nontoxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions described herein may be orally administered in any orally acceptable dosage form including, but not limited to, capsules, tablets, aqueous suspensions or solutions. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions described herein may be administered in the form of suppositories for rectal administration. These can be prepared by mixing the agent with a suitable non-irritating excipient which is solid at room temperature but liquid at rectal temperature and therefore will melt in the rectum to release the drug. Such materials include, but are not limited to, cocoa butter, beeswax and polyethylene glycols.

The pharmaceutical compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutical compositions may be formulated as micronized suspensions in isotonic, pH adjusted sterile saline, or, specifically, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions may also be administered to the respiratory tract. The respiratory tract includes the upper airways, including the oropharynx and larynx, followed by the lower airways, which include the trachea followed by bifurcations into the bronchi and bronchioli. Pulmonary delivery compositions can be delivered by inhalation by the patient of a dispersion so that the active ingredient within the dispersion can reach the lung where it can, for example, be readily absorbed through the alveolar region directly into blood circulation. Pulmonary delivery can be achieved by different approaches, including the use of nebulized, aerosolized, micellular and dry powder-based formulations; administration by inhalation may be oral and/or nasal. Delivery can be achieved with liquid nebulizers, aerosol-based inhalers, and dry powder dispersion devices. Metered-dose devices are preferred. One of the benefits of using an atomizer or inhaler is that the potential for contamination is minimized because the devices are self contained. Dry powder dispersion devices, for example, deliver drugs that may be readily formulated as dry powders. A pharmaceutical composition of the invention may be stably stored as lyophilized or spray-dried powders by itself or in combination with suitable powder carriers. The delivery of a pharmaceutical composition of the invention for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament. Examples of pharmaceutical devices for aerosol delivery include metered dose inhalers (MDIs), dry powder inhalers (DPIs), and air-jet nebulizers.

The compounds for use in the methods of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

Methods for the Productions of Vaccines:

As described above, the methods of the present invention are particularly useful for screening a plurality of substances that may be used for the production of influenza virus vaccines. Actually, substances capable of increasing the replication capacity of an influenza virus may be used for the production of vaccines.

Typically, methods for producing vaccines comprise the steps consisting of i) infecting said cell with an influenza virus and ii) culturing said infected cell with a substance capable of increasing the replication capacity of an influenza virus and iii) and recovering the resulting replicated virus.

Following culture for a suitable period of time to permit replication of the virus to high titer, the virus can be indeed recovered. Viruses can typically be recovered from the culture medium, in which infected (transfected) cells have been grown. Typically crude medium is clarified prior to concentration of influenza viruses. Common methods include filtration, ultrafiltration, adsorption on barium sulfate and elution, and centrifugation. For example, crude medium from infected cultures can first be clarified by centrifugation at, e.g., 1000-2000×g for a time sufficient to remove cell debris and other large particulate matter, e.g., between 10 and 30 minutes. Alternatively, the medium is filtered through a 0.8 um cellulose acetate filter to remove intact cells and other large particulate matter. Optionally, the clarified medium supernatant is then centrifuged to pellet the influenza viruses, e.g., at 15,000×g, for approximately 3-5 hours. Following resuspension of the virus pellet in an appropriate buffer, such as STE (0.01 MTris-HC1; 0.15MNaC1; 0.0001 MEDTA) or phosphate buffered saline (PBS) at pH 7.4, the virus is concentrated by density gradient centrifugation on sucrose (60%12%) or potassium tartrate (50%-10%). Either continuous or step gradients, e.g., a sucrose gradient between 12% and 60% in four 12% steps, are suitable. The gradients are centrifuged at a speed, and for a time, sufficient for the viruses to concentrate into a visible band for recovery. Alternatively, and for most large scale commercial applications, virus is elutriated from density gradients using a zonal-centrifuge rotor operating in continuous mode. Additional details sufficient to guide one of skill through the preparation of influenza viruses from tissue culture are provided, e.g., in Furminger. Vaccine Production, in Nicholson et al. (eds) Textbook of Influenza pp. 324-332; Merten et al. (1996) Production of influenza virus in cell culturesfor vaccine preparation, in Cohen & Shafferman (eds) Novel Strategies in Design and Production of Vaccines pp. 141-151, and U.S. Pat. No. 5,690,937, U.S. publication application nos. 20040265987, 20050266026 and 20050158342, which are incorporated by reference herein. If desired, the recovered viruses can be stored at −80° C. in the presence of sucrose-phosphate-glutamate (SPG) as a stabilizer.

The resulting replicated virus can be indeed concentrated as above described and then be inactivated or attenuated using any method well known in the art.

Inactivated influenza virus vaccines of the invention are typically provided by inactivating replicated virus of the invention using known methods, such as, but not limited to, formalin or .beta.-propiolactone treatment. Inactivated vaccine types that can be used in the invention can include whole-virus (WV) vaccine or subvirion (SV) virus vaccine. The WV vaccine contains intact, inactivated virus, while the SV vaccine contains purified virus disrupted with detergents that solubilize the lipid-containing viral envelope, followed by chemical inactivation of residual virus.

In addition, vaccines that can be used include those containing the isolated HA and NA surface proteins, which are referred to as surface antigen vaccines. In general, the responses to SV and surface antigen (i.e., purified HA or NA) vaccines are similar. An experimental inactivated WV vaccine containing an NA antigen immunologically related to the epidemic virus and an unrelated HA appears to be less effective than conventional vaccines. Inactivated vaccines containing both relevant surface antigens are preferred.

Live, attenuated influenza virus vaccines, using replicated virus of the invention, can also be used for preventing or treating influenza virus infection, according to known method steps: Attenuation is preferably achieved in a single step by transfer of attenuating genes from an attenuated donor virus to a replicated isolate or reassorted virus according to known methods (see, e.g., Murphy, Infect. Dis. Clin. Pract. 2:174-181 (1993)). Since resistance to influenza A virus is mediated by the development of an immune response to the HA and NA glycoproteins, the genes coding for these surface antigens must come from the reassorted viruses or high growth clinical isolates. The attenuating genes are derived from the attenuated parent. In this approach, genes that confer attenuation preferably do not code for the HA and NA glycoproteins. Otherwise, these genes could not be transferred to reassortants bearing the surface antigens of the clinical virus isolate.

Many donor viruses have been evaluated for their ability to reproducibly attenuate influenza viruses. As a non-limiting example, the A/Ann Arbor(AA)/6/60 (H2N2) cold adapted (ca) donor virus can be used for attenuated vaccine production (see, e.g., Edwards, J. Infect. Dis. 169:68-76 (1994); Murphy, Infect. Dis. Clin. Pract. 2:174-181 (1993)). Additionally, live, attenuated reassortant virus vaccines can be generated by mating the donor virus with a virulent replicated virus of the invention. Reassortant progeny are then selected at 25° C. (restrictive for replication of virulent virus), in the presence of an H2N₂ antiserum, which inhibits replication of the viruses bearing the surface antigens of the attenuated A/AA/6/60 (H2N2) ca donor virus.

A large series of H1N1 and H3N₂ reassortants have been evaluated in humans and found to be satisfactorily: (a) infectious, (b) attenuated for seronegative children and immunologically primed adults, (c) immunogenic and (d) genetically stable. The immunogenicity of the ca reassortants parallels their level of replication. Thus, the acquisition of the six transferable genes of the ca donor virus by new wild-type viruses has reproducibly attenuated these viruses for use in vaccinating susceptible adults and children.

Other attenuating mutations can be introduced into influenza virus genes by site-directed mutagenesis to rescue infectious viruses bearing these mutant genes. Attenuating mutations can be introduced into non-coding regions of the genome, as-well as into coding regions. Such attenuating mutations can also be introduced into genes other than the HA or NA, e.g., the PB2 polymerase gene (Subbarao et al., J. Virol. 67:7223-7228 (1993)). Thus, new donor viruses can also be generated bearing attenuating mutations introduced by site-directed mutagenesis, and such new donor viruses can be used in the production of live attenuated reassortants H1N1 and H3N₂ vaccine candidates in a manner analogous to that described above for the A/AA/6/60 ca donor virus. Similarly, other known and suitable attenuated donor strains can be reassorted with replicated influenza virus of the invention to obtain attenuated vaccines suitable for use in the vaccination of mammals. (Ewami et al., Proc. Natl. Acad. Sci. USA 87:3802-3805 (1990); Muster et al., Proc. Natl. Acad. Sci. USA 88:5177-5181 (1991); Subbarao et al., J. Virol. 67:7223-7228 (1993); U.S. patent application Ser. No. 08/471,100, which references are entirely incorporated by reference)

It is preferred that such attenuated viruses maintain the genes from the replicated virus that encode antigenic determinants substantially similar to those of the original clinical isolates. This is because the purpose of the attenuated vaccine is to provide substantially the same antigenicity as the original clinical isolate of the virus, while at the same time lacking infectivity to the degree that the vaccine causes minimal chance of inducing a serious pathogenic condition in the vaccinated mammal.

The replicated virus that is attenuated or inactivated may be then formulated in a vaccine composition.

Vaccine compositions of the present invention, suitable for inoculation or for parenteral or oral administration, comprise attenuated or inactivated influenza viruses, optionally further comprising sterile aqueous or non-aqueous solutions, suspensions, and emulsions. The composition can further comprise auxiliary agents or excipients, as known in the art.

Preparations for parenteral administration include sterile aqueous or non-aqueous solutions, suspensions, and/or emulsions, which may contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents. See, e.g., Berkow, infra, Goodman, infra, Avery's, infra, Osol, infra and Katzung, infra, which are entirely incorporated herein by reference, included all references cited therein.

When a vaccine composition of the present invention is used for administration to an individual, it can further comprise salts, buffers, adjuvants, or other substances which are desirable for improving the efficacy of the composition.

Adjuvants are substances that can be used to augment a specific immune response. Normally, the adjuvant and the composition are mixed prior to presentation to the immune system, or presented separately, but into the same site of the mammal being immunized.

Heterogeneity in the vaccine may be provided by mixing replicated influenza viruses for at least two influenza virus strains, such as 2-50 strains or any range or value therein. Influenza A or B virus strains having a modem antigenic composition are preferred. According to the present invention, vaccines can be provided for variations in a single strain of an influenza virus or for more than one strain of influenza viruses, using techniques known in the art.

Once prepared the vaccine composition may be then administered in a subject in need thereof. Typically, an attenuated or inactivated vaccine composition of the present invention may thus be provided either before the onset of infection (so as to prevent or attenuate an anticipated infection) or after the initiation of an actual infection. For example, administration of such a vaccine composition may be by various parenteral routes such as subcutaneous, intravenous, intradermal, intramuscular, intraperitoneal, intranasal, oral or transdermal routes. Parenteral administration can be by bolus injection or by gradual perfusion over time. A preferred mode of using a vaccine composition of the present invention is by intramuscular or subcutaneous application. See, e.g., Berkow, infra, Goodman, infra, Avery, infra and Katzung, infra, which are entirely incorporated herein by reference, including all references cited therein.

The vaccine composition is administered to the subject in a effective amount. According to the present invention, an “effective amount” of a vaccine composition is one that is sufficient to achieve a desired biological effect. It is understood that the effective dosage will be dependent upon the age, sex, health, and weight of the recipient, kind of concurrent treatment, if any, frequency of treatment, and the nature of the effect wanted. The ranges of effective doses provided below are not intended to limit the invention and represent preferred dose ranges. However, the most preferred dosage will be tailored to the individual subject, as is understood and determinable by one of skill in the art, without undue experimentation.

The invention will be further illustrated by the following figures and examples. However, these examples and figures should not be interpreted in any way as limiting the scope of the present invention.

### Example

Methods

Construction of the Influenza ORFeome: The Influenza genome is composed of eight single stranded RNA molecules encoding eleven proteins (HA, NA, NP, M1, M2, NS1, NEP, PA, PB1, PB1-F2, PB2). All 11 open reading frames from several Influenza A viruses (A/Puerto-Rico/8/34, A/WSN33/1933 T S61, A/Lyon/712/06, A/Poitiers/484/05, A/Chicken/Scotland/59, A/Turkey/582/2006, A/Vietnam/1194/2003, A/Chicken/Belgium/2003, A/Equine/Prague/56, A/Chicken/HK/69/97, A//HK/1073/97, A/Duck/Australia/348/83) were cloned in a Gateway recombinational cloning system. Each ORF was PCR amplified (with KOD polymerase, Novagen) using attB1. 1 and attB2.1 recombination sites fused to forward and reverse primers, then cloned into pDONR223 (6). All entry clones were sequence verified.

Yeast Two hybrid (Y2H) library screens and yeast two hybrid matrix: Influenza ORFs were transferred from pDONR223 into bait vector (pPC97) to be expressed as Ga1W-DB fusions in yeast. Because bait constructs sometimes self-transactivate reporter genes, SD-L-H culture medium were supplemented with 3-aminotriazole (3-AT). Appropriate concentrations of this drug were determined by growing bait strains on SD-L-H medium supplemented with increasing concentrations of 3-AT. Screens were performed by yeast mating, using AH109 and Y187 yeast strains (Clontech (S)). Bait vectors were transformed into AH109 (bait strain) and human spleen, fetal brain and respiratory epithelium AD-cDNA libraries (Invitrogen) were transformed into Y187 (prey strain). Single bait strains were mated with prey strains then diploids were plated on SD-W-L-H+3-AT medium. Positive clones were maintained onto this selective medium for 15 days to eliminate any contaminant AD-cDNA plasmid (9). AD-cDNAs were PCR amplified and inserts were sequenced.

Pair wise yeast two hybrid interaction analyses were performed by yeast mating, using Y187 and AH109 yeasts strains (Clontech #15604093) for NS1 and NS2 proteins. NS1 and NS2 cellular target genes were transferred from pDONR207 into a prey vector (pPC86, Invitrogen) to be expressed as Ga14-DNA Binding domain fusions in yeast. Preys and baits (NS1 and NS2 cloned in pPC97) vectors were transformed into Y187 and AH109. Bait and prey strains were mated and plated on a selective medium lacking histidine and supplemented with 5 mM 3-amino-triazole to test the interaction-dependent transactivation of HIS3 reporter gene.

Results:

All the interactions identified by the above described methods are reported in table 1.

TABLE 1
interactions identified between human host
cell proteins and influenza viral proteins

| | | | interaction | | activity |
|---|---|---|---|---|---|
| | | | also | | also |
| Ensembl | Gene | Influenza | reported in | | reported in |
| Gene Id | Name | protein | literature | activity? | literature |
| ENSG00000119688 | ABCD4 | NS1 | | | |
| ENSG00000119688 | ABCD4 | NS2 | | | |
| ENSG00000072110 | ACTN1 | M1 | | | |
| ENSG00000160710 | ADAR | NS1 | | | |
| ENSG00000106305 | AIMP2 | NS2 | x | gene having | |
| | | | | antiviral | |
| | | | | activity | |
| ENSG00000144908 | ALDH1L1 | PB2 | | | |
| ENSG00000159712 | ANKRD18B | PB2 | | | |
| ENSG00000006125 | AP2B1 | NS1 | | | |
| ENSG00000006125 | AP2B1 | NS2 | | | |
| ENSG00000168374 | ARF4 | NS2 | | | |
| ENSG00000143437 | ARNT | NS2 | | | |
| ENSG00000143437 | ARNT | NS1 | | | |
| ENSG00000171456 | ASXL1 | PB2 | | | |
| ENSG00000171681 | ATF7IP | NS1 | | | |
| ENSG00000143153 | ATP1B1 | PB2 | | | |
| ENSG00000116459 | ATP5F1 | NS1 | | gene having | x |
| | | | | proviral | |
| | | | | activity | |
| ENSG00000136888 | ATP6V1G1 | PA | | gene having | x |
| | | | | proviral | |
| | | | | activity | |
| ENSG00000136888 | ATP6V1G1 | NS2 | | gene having | x |
| | | | | proviral | |
| | | | | activity | |
| ENSG00000136888 | ATP6V1G1 | NS1 | | gene having | x |
| | | | | proviral | |
| | | | | activity | |
| ENSG00000168646 | AXIN2 | NS1 | | gene having | |
| | | | | antiviral | |
| | | | | activity | |
| ENSG00000002330 | BAD | NS1 | | gene having | |
| | | | | antiviral | |
| | | | | activity | |
| ENSG00000030110 | BAK1 | NS1 | | | |
| ENSG00000087088 | BAX | NS1 | | gene having | |
| | | | | proviral | |
| | | | | activity | |
| ENSG00000171552 | BCL2L1 | NS1 | | gene having | |
| | | | | antiviral | |
| | | | | activity | |
| ENSG00000153094 | BCL2L11 | NS1 | | gene having | |
| | | | | proviral | |
| | | | | activity | |
| ENSG00000029363 | BCLAF1 | NS1 | | gene having | |
| | | | | antiviral | |
| | | | | activity | |
| ENSG00000029363 | BCLAF1 | NS2 | | gene having | |
| | | | | antiviral | |
| | | | | activity | |
| ENSG00000186716 | BCR | NS2 | | | |
| ENSG00000186716 | BCR | PB2 | | | |
| ENSG00000198908 | BHLHB9 | NS1 | | gene having | |
| | | | | antiviral | |
| | | | | activity | |
| ENSG00000198908 | BHLHB9 | NS2 | | gene having | |
| | | | | antiviral | |
| | | | | activity | |
| ENSG00000015475 | BID | NS1 | | gene having | x |
| | | | | antiviral | |
| | | | | activity | |
| ENSG00000136717 | BIN1 | NS1 | | | |
| ENSG00000172331 | BPGM | NS1 | | | |
| ENSG00000172331 | BPGM | NS2 | | | |
| ENSG00000157764 | BRAF | NS1 | | gene having | |
| | | | | proviral | |
| | | | | activity | |
| ENSG00000119411 | BSPRY | NS2 | | | |
| ENSG00000087302 | C14orf166 | NS1 | | | |
| ENSG00000087302 | C14orf166 | PA | x | | |
| ENSG00000125999 | C20orf114 | PB2 | | | |
| ENSG00000112936 | C7 | PA | | | |
| ENSG00000204711 | C9orf135 | PB2 | | | |
| ENSG00000136819 | C9orf78 | NS2 | | | |
| ENSG00000136436 | CALCOCO2 | PB1-F2 | | | |
| ENSG00000131236 | CAP1 | PB2 | | | |
| ENSG00000131236 | CAP1 | NS1 | | | |
| ENSG00000162909 | CAPN2 | PB2 | | | |
| ENSG00000187796 | CARD9 | NS2 | | gene having | |
| | | | | antiviral | |
| | | | | activity | |
| ENSG00000110619 | CARS | NS1 | | gene having | |
| | | | | proviral | |
| | | | | activity | |
| ENSG00000141582 | CBX4 | NS1 | | | |
| ENSG00000159214 | CCDC24 | NS2 | | gene having | |
| | | | | proviral | |
| | | | | activity | |
| ENSG00000204536 | CCHCR1 | NS2 | | | |
| ENSG00000204536 | CCHCR1 | NS1 | | | |
| ENSG00000108691 | CCL2 | NS1 | | | |
| ENSG00000128845 | CCPG1 | NS1 | | | |
| ENSG00000128845 | CCPG1 | NS2 | | | |
| ENSG00000019582 | CD74 | PB1 | | | |
| ENSG00000101224 | CDC25B | NS1 | | | |
| ENSG00000189229 | CDK11B | NS1 | | | |
| ENSG00000145241 | CENPC1 | NS1 | | | |
| ENSG00000153044 | CENPH | NS2 | | | |
| ENSG00000119397 | CEP110 | NS2 | | | |
| ENSG00000119397 | CEP110 | NS1 | | | |
| ENSG00000101639 | CEP192 | NS2 | | | |
| ENSG00000196959 | CES1 | PB2 | | | |
| ENSG00000167670 | CHAF1A | PB2 | | | |
| ENSG00000125611 | CHCHD5 | NS2 | | gene having | |
| | | | | proviral | |
| | | | | activity | |
| ENSG00000170004 | CHD3 | NS1 | | | |
| ENSG00000124177 | CHD6 | PA | | | |
| ENSG00000107566 | CHUK | NS1 | | | |
| ENSG00000204418 | CLIC1 | NS2 | | | |
| ENSG00000162368 | CMPK | NS2 | | | |
| ENSG00000162368 | CMPK | NS1 | | | |
| ENSG00000133103 | COG6 | NS1 | | gene having | |
| | | | | antiviral | |
| | | | | activity | |
| ENSG00000133103 | COG6 | NS2 | | gene having | |
| | | | | antiviral | |
| | | | | activity | |
| ENSG00000157312 | COG8 | NS2 | | gene having | |
| | | | | antiviral | |
| | | | | activity | |
| ENSG00000157312 | COG8 | NS1 | | gene having | |
| | | | | antiviral | |
| | | | | activity | |
| ENSG00000173163 | COMMD1 | PB2 | | | |
| ENSG00000173163 | COMMD1 | NS2 | | | |
| ENSG00000173163 | COMMD1 | PA | | | |
| ENSG00000173163 | COMMD1 | NS1 | | | |
| ENSG00000121022 | COPS5 | PB2 | | | |
| ENSG00000127054 | CPSF3L | NS1 | | gene having | |
| | | | | antiviral | |
| | | | | activity | |
| ENSG00000160917 | CPSF4 | NS1 | x | gene having | |
| | | | | proviral | |
| | | | | activity | |
| ENSG00000160917 | CPSF4 | NS2 | | gene having | |
| | | | | proviral | |
| | | | | activity | |
| ENSG00000167193 | CRK | NS1 | x | | |
| ENSG00000100055 | CYTH4 | NS2 | | | |
| ENSG00000183283 | DAZAP2 | PB1 | | | |
| ENSG00000204843 | DCTN1 | PB2 | | | |
| ENSG00000175203 | DCTN2 | NS2 | | | |
| ENSG00000136271 | DDX56 | NS1 | | | |
| ENSG00000132535 | DLG4 | NS1 | | gene having | |
| | | | | antiviral | |
| | | | | activity | |
| ENSG00000132002 | DNAJB1 | NS2 | | | |
| ENSG00000132002 | DNAJB1 | PB2 | | | |
| ENSG00000187726 | DNAJB13 | PB2 | | | |
| ENSG00000151914 | DST | PA | | | |
| ENSG00000167264 | DUS2L | NS1 | | gene having | |
| | | | | antiviral | |
| | | | | activity | |
| ENSG00000088986 | DYNLL1 | NS2 | | gene having | |
| | | | | proviral | |
| | | | | activity | |
| ENSG00000088986 | DYNLL1 | NS1 | | gene having | |
| | | | | proviral | |
| | | | | activity | |
| ENSG00000179151 | EDC3 | NS1 | | gene having | |
| | | | | proviral | |
| | | | | activity | |
| ENSG00000107223 | EDF1 | NS1 | | | |
| ENSG00000156508 | EEF1A1 | NS1 | | gene having | x |
| | | | | proviral | |
| | | | | activity | |
| ENSG00000104529 | EEF1D | PA | | | |
| ENSG00000100129 | EIF3S6IP | PB2 | | | |
| ENSG00000074800 | ENO1 | NS2 | | gene having | |
| | | | | antiviral | |
| | | | | activity | |
| ENSG00000074800 | ENO1 | NS1 | | gene having | |
| | | | | antiviral | |
| | | | | activity | |
| ENSG00000136628 | EPRS | NS2 | | | |
| ENSG00000112851 | ERBB2IP | NS1 | | gene having | |
| | | | | antiviral | |
| | | | | activity | |
| ENSG00000112851 | ERBB2IP | NS2 | | gene having | |
| | | | | antiviral | |
| | | | | activity | |
| ENSG00000166595 | FAM96B | PB2 | | | |
| ENSG00000165281 | FANCG | PB2 | | | |
| ENSG00000137312 | FLOT1 | PB2 | | | |
| ENSG00000115414 | FN1 | PB1-F2 | | | |
| ENSG00000049768 | FOXP3 | NS1 | | | |
| ENSG00000089280 | FUS | PB1 | | | |
| ENSG00000132139 | GAS2L2 | NS2 | | | |
| ENSG00000123159 | GIPC1 | NS1 | | | |
| ENSG00000123159 | GIPC1 | NS2 | | | |
| ENSG00000135821 | GLUL | PB2 | | | |
| ENSG00000140632 | GLYR1 | NS2 | | gene having | |
| | | | | antiviral | |
| | | | | activity | |
| ENSG00000140632 | GLYR1 | PA | | gene having | |
| | | | | antiviral | |
| | | | | activity | |
| ENSG00000140632 | GLYR1 | NS1 | | gene having | |
| | | | | antiviral | |
| | | | | activity | |
| ENSG00000162419 | GMEB1 | NS1 | | gene having | x |
| | | | | proviral | |
| | | | | activity | |
| ENSG00000162419 | GMEB1 | NS2 | | gene having | x |
| | | | | proviral | |
| | | | | activity | |
| ENSG00000132522 | GPS2 | NS2 | | | |
| ENSG00000132522 | GPS2 | NS1 | | | |
| ENSG00000148180 | GSN | PA | | gene having | |
| | | | | proviral | |
| | | | | activity | |
| ENSG00000148180 | GSN | NS1 | | gene having | |
| | | | | proviral | |
| | | | | activity | |
| ENSG00000095951 | HIVEP1 | M1 | | gene having | |
| | | | | antiviral | |
| | | | | activity | |
| ENSG00000095951 | HIVEP1 | NS1 | | gene having | |
| | | | | antiviral | |
| | | | | activity | |
| ENSG00000095951 | HIVEP1 | NS2 | | gene having | |
| | | | | antiviral | |
| | | | | activity | |
| ENSG00000168000 | HNRPUL2 | NS2 | | | |
| ENSG00000166598 | HSP90B1 | NS2 | | | |
| ENSG00000115317 | HTRA2 | PB2 | | | |
| ENSG00000086758 | HUWE1 | NS2 | | | |
| ENSG00000211896 | IGHG1 | NS1 | | | |
| ENSG00000211896 | IGHG1 | NS2 | | | |
| ENSG00000211893 | IGHG2 | NS2 | | | |
| ENSG00000211899 | IGHM | PB1 | | | |
| ENSG00000211620 | IGKV1D | NS1 | | | |
| ENSG00000113141 | IK | PB2 | | | |
| ENSG00000143466 | IKBKE | NS1 | | | |
| ENSG00000110324 | IL10RA | NS1 | | gene having | |
| | | | | proviral | |
| | | | | activity | |
| ENSG00000129351 | ILF3 | NS1 | | | |
| ENSG00000129351 | ILF3 | NS2 | | | |
| ENSG00000111653 | ING4 | NS2 | | | |
| ENSG00000143164 | IQWD1 | PB2 | | | |
| ENSG00000169047 | IRS1 | NS1 | | | |
| ENSG00000185950 | IRS2 | NS1 | | | |
| ENSG00000189337 | KAZRIN | NS1 | | | |
| ENSG00000168301 | KCTD6 | NS2 | | gene having | |
| | | | | proviral | |
| | | | | activity | |
| ENSG00000168301 | KCTD6 | NS1 | | gene having | |
| | | | | proviral | |
| | | | | activity | |
| ENSG00000121774 | KHDRBS1 | NP | | | |
| ENSG00000112624 | KIAA0240 | NS2 | | | |
| ENSG00000100578 | KIAA0586 | PA | | | |
| ENSG00000174501 | KIAA1641 | PB2 | | | |
| ENSG00000091136 | LAMB1 | NS2 | | | |
| ENSG00000050555 | LAMC3 | NS2 | | | |
| ENSG00000155506 | LARP1 | NS1 | | gene having | x |
| | | | | proviral | |
| | | | | activity | |
| ENSG00000111716 | LDHB | NS1 | | | |
| ENSG00000111716 | LDHB | NS2 | | | |
| ENSG00000131981 | LGALS3 | NS1 | | | |
| ENSG00000033122 | LRRC7 | NS1 | | | |
| ENSG00000133739 | LRRCC1 | NS1 | | | |
| ENSG00000124831 | LRRFIP1 | PA | | | |
| ENSG00000198909 | MAP3K3 | NS1 | | gene having | |
| | | | | antiviral | |
| | | | | activity | |
| ENSG00000050748 | MAPK9 | NS1 | x | gene having | |
| | | | | antiviral | |
| | | | | activity | |
| ENSG00000151332 | MBIP | NS2 | | gene having | |
| | | | | antiviral | |
| | | | | activity | |
| ENSG00000125885 | MCM8 | PB2 | | | |
| ENSG00000020426 | MNAT1 | PB2 | | | |
| ENSG00000178802 | MPI | NP | | | |
| ENSG00000037757 | MRI1 | PA | | | |
| ENSG00000198727 | MT-CYB | NS1 | | | |
| ENSG00000013364 | MVP | PB2 | | | |
| ENSG00000136997 | MYC | PB2 | | | |
| ENSG00000114503 | NCBP2 | NS2 | | | |
| ENSG00000115053 | NCL | NS2 | | | |
| ENSG00000111912 | NCOA7 | PB2 | | | |
| ENSG00000100503 | NIN | NS2 | | gene having | |
| | | | | antiviral | |
| | | | | activity | |
| ENSG00000186416 | NKRF | NS1 | | | |
| ENSG00000123609 | NMI | PB2 | | | |
| ENSG00000103512 | NOMO1 | NS2 | | | |
| ENSG00000185164 | NOMO2 | PA | | | |
| ENSG00000185164 | NOMO2 | PB2 | | | |
| ENSG00000124588 | NQO2 | NS2 | | gene having | |
| | | | | antiviral | |
| | | | | activity | |
| ENSG00000124588 | NQO2 | NS1 | | gene having | |
| | | | | antiviral | |
| | | | | activity | |
| ENSG00000124588 | NQO2 | PA | | gene having | |
| | | | | antiviral | |
| | | | | activity | |
| ENSG00000185551 | NR2F2 | NS1 | | | |
| ENSG00000107672 | NSMCE4A | PB2 | | | |
| ENSG00000124789 | NUP153 | NS2 | | | |
| ENSG00000126883 | NUP214 | NS2 | | | |
| ENSG00000126883 | NUP214 | PA | | | |
| ENSG00000166228 | PCBD1 | PA | | | |
| ENSG00000169564 | PCBP1 | NS1 | | | |
| ENSG00000169564 | PCBP1 | PA | | | |
| ENSG00000175198 | PCCA | PB2 | | | |
| ENSG00000132646 | PCNA | PB2 | | | |
| ENSG00000079739 | PGM1 | PB2 | | | |
| ENSG00000143393 | PI4KB | NS1 | | | |
| ENSG00000033800 | PIAS1 | NS1 | | | |
| ENSG00000078043 | PIAS2 | NS1 | | | |
| ENSG00000131788 | PIAS3 | NS1 | | gene having | |
| | | | | antiviral | |
| | | | | activity | |
| ENSG00000131788 | PIAS3 | NS2 | | gene having | |
| | | | | antiviral | |
| | | | | activity | |
| ENSG00000145675 | PIK3R1 | NS1 | x | gene having | |
| | | | | antiviral | |
| | | | | activity | |
| ENSG00000105647 | PIK3R2 | NS1 | x | gene having | x |
| | | | | antiviral | |
| | | | | activity | |
| ENSG00000116711 | PLA2G4A | PB2 | | | |
| ENSG00000187091 | PLCD1 | NS2 | | | |
| ENSG00000106628 | POLD2 | NS2 | | gene having | |
| | | | | antiviral | |
| | | | | activity | |
| ENSG00000106628 | POLD2 | NS1 | | gene having | |
| | | | | antiviral | |
| | | | | activity | |
| ENSG00000181222 | POLR2A | PA | x | | |
| ENSG00000047315 | POLR2B | PB2 | | | |
| ENSG00000132664 | POLR3F | NS2 | | | |
| ENSG00000132664 | POLR3F | NS1 | | | |
| ENSG00000132664 | POLR3F | PB2 | | | |
| ENSG00000135213 | POM121 | NS2 | | | |
| ENSG00000135213 | POM121 | NS1 | | | |
| ENSG00000132963 | POMP | NS2 | | gene having | |
| | | | | antiviral | |
| | | | | activity | |
| ENSG00000132963 | POMP | PB2 | | gene having | |
| | | | | antiviral | |
| | | | | activity | |
| ENSG00000169230 | PRELID1 | NS2 | | gene having | |
| | | | | antiviral | |
| | | | | activity | |
| ENSG00000138078 | PREPL | NS1 | | gene having | |
| | | | | antiviral | |
| | | | | activity | |
| ENSG00000138078 | PREPL | NS2 | | gene having | |
| | | | | antiviral | |
| | | | | activity | |
| ENSG00000180228 | PRKRA | NS1 | x | gene having | |
| | | | | antiviral | |
| | | | | activity | |
| ENSG00000101182 | PSMA7 | PB2 | | | |
| ENSG00000101182 | PSMA7 | PA | | | |
| ENSG00000101182 | PSMA7 | NS2 | | | |
| ENSG00000101182 | PSMA7 | NS1 | | | |
| ENSG00000008018 | PSMB1 | NS2 | | | |
| ENSG00000165916 | PSMC3 | PB1-F2 | | | |
| ENSG00000099341 | PSMD8 | PB2 | | | |
| ENSG00000132155 | RAF1 | NS1 | | gene having | |
| | | | | proviral | |
| | | | | activity | |
| ENSG00000116191 | RALGPS2 | NS1 | | | |
| ENSG00000125826 | RBCK1 | NS1 | | | |
| ENSG00000125826 | RBCK1 | NS2 | | | |
| ENSG00000136104 | RNASEH2B | NS1 | | | |
| ENSG00000147403 | RPL10 | NS2 | | | |
| ENSG00000147403 | RPL10 | NS1 | | | |
| ENSG00000142676 | RPL11 | PB1 | | | |
| ENSG00000197958 | RPL12 | PB2 | | | |
| ENSG00000142541 | RPL13A | NS1 | | gene having | x |
| | | | | proviral | |
| | | | | activity | |
| ENSG00000198242 | RPL23A | NS1 | | gene having | |
| | | | | antiviral | |
| | | | | activity | |
| ENSG00000156482 | RPL30 | NS1 | | | |
| ENSG00000109475 | RPL34 | NS2 | | gene having | |
| | | | | proviral | |
| | | | | activity | |
| ENSG00000161016 | RPL8 | PB1 | | | |
| ENSG00000177600 | RPLP2 | NS1 | | | |
| ENSG00000149273 | RPS3 | NS1 | | gene having | |
| | | | | proviral | |
| | | | | activity | |
| ENSG00000170889 | RPS9 | PB1 | | | |
| ENSG00000168028 | RPSA | NS2 | | gene having | |
| | | | | proviral | |
| | | | | activity | |
| ENSG00000180900 | SCRIB | NS1 | | gene having | |
| | | | | proviral | |
| | | | | activity | |
| ENSG00000117118 | SDHB | NS2 | | gene having | |
| | | | | antiviral | |
| | | | | activity | |
| ENSG00000117118 | SDHB | NS1 | | gene having | |
| | | | | antiviral | |
| | | | | activity | |
| ENSG00000175793 | SFN | NS1 | | | |
| ENSG00000175793 | SFN | NS2 | | | |
| ENSG00000116560 | SFPQ | NS1 | | | |
| ENSG00000160691 | SHC1 | NS1 | | | |
| ENSG00000113558 | SKP1A | NS2 | | | |
| ENSG00000165449 | SLC16A9 | PB1 | | | |
| ENSG00000169100 | SLC25A6 | NS2 | | gene having | |
| | | | | proviral | |
| | | | | activity | |
| ENSG00000176463 | SLCO3A1 | NS1 | | | |
| ENSG00000124107 | SLPI | PA | | | |
| ENSG00000099956 | SMARCB1 | PB2 | | | |
| ENSG00000143553 | SNAPAP | PB2 | | | |
| ENSG00000164975 | SNAPC3 | NS2 | | | |
| ENSG00000164975 | SNAPC3 | NS1 | | | |
| ENSG00000165684 | SNAPC4 | NS2 | | | |
| ENSG00000165684 | SNAPC4 | PA | | | |
| ENSG00000165684 | SNAPC4 | NS1 | | | |
| ENSG00000114520 | SNX4 | NS1 | | gene having | |
| | | | | proviral | |
| | | | | activity | |
| ENSG00000114520 | SNX4 | NS2 | | gene having | |
| | | | | proviral | |
| | | | | activity | |
| ENSG00000089006 | SNX5 | NS2 | | | |
| ENSG00000159140 | SON | NS1 | | gene having | x |
| | | | | proviral | |
| | | | | activity | |
| ENSG00000198513 | SPG3A | NP | | | |
| ENSG00000124214 | STAU1 | NS1 | x | | |
| ENSG00000040341 | STAU2 | NS1 | | gene having | |
| | | | | proviral | |
| | | | | activity | |
| ENSG00000040341 | STAU2 | M1 | | gene having | |
| | | | | proviral | |
| | | | | activity | |
| ENSG00000040341 | STAU2 | NS2 | | gene having | |
| | | | | proviral | |
| | | | | activity | |
| ENSG00000165209 | STRBP | NS1 | | | |
| ENSG00000116030 | SUMO1 | NS1 | x | gene having | x |
| | | | | proviral | |
| | | | | activity | |
| ENSG00000147642 | SYBU | M1 | | | |
| ENSG00000147133 | TAF1 | NS1 | | gene having | |
| | | | | antiviral | |
| | | | | activity | |
| ENSG00000136560 | TANK | NS1 | | | |
| ENSG00000139546 | TARBP2 | NS1 | x | | |
| ENSG00000083454 | TAX1BP3 | NS2 | | | |
| ENSG00000083454 | TAX1BP3 | NS1 | | | |
| ENSG00000109736 | TETRAN | NS2 | | | |
| ENSG00000109736 | TETRAN | PB2 | | | |
| ENSG00000182646 | TMEM29 | PB2 | | | |
| ENSG00000129991 | TNNI3 | NS2 | | gene having | |
| | | | | proviral | |
| | | | | activity | |
| ENSG00000129991 | TNNI3 | NS1 | | gene having | |
| | | | | proviral | |
| | | | | activity | |
| ENSG00000103197 | TSC2 | NS1 | | | |
| ENSG00000149292 | TTC12 | PB2 | | | |
| ENSG00000115514 | TXNDC9 | NS2 | | | |
| ENSG00000170315 | UBB | NS2 | | | |
| ENSG00000150991 | UBC | NS2 | | | |
| ENSG00000150991 | UBC | NS1 | | | |
| ENSG00000150991 | UBC | PB2 | | | |
| ENSG00000103275 | UBE2I | NS1 | | | |
| ENSG00000175564 | UCP3 | NS1 | | | |
| ENSG00000138592 | USP8 | NS1 | | | |
| ENSG00000115652 | UXS1 | PA | | | |
| ENSG00000103043 | VAC 14 | NS1 | | | |
| ENSG00000103043 | VAC 14 | NS2 | | | |
| ENSG00000116809 | ZBTB17 | NS2 | | | |
| ENSG00000116809 | ZBTB17 | NS1 | | | |
| ENSG00000121766 | ZCCHC17 | PB2 | | | |
| ENSG00000172667 | ZMAT3 | NS1 | | | |
| ENSG00000121741 | ZMYM2 | NS2 | | | |
| ENSG00000121741 | ZMYM2 | NS1 | | | |
| ENSG00000198169 | ZNF251 | PB2 | | | |
| ENSG00000198169 | ZNF251 | NS2 | | | |
| ENSG00000198169 | ZNF251 | NS1 | | | |
| ENSG00000198522 | ZNF512 | NS1 | | gene having | |
| | | | | antiviral | |
| | | | | activity | |
| ENSG00000167395 | ZNF646 | NS1 | | | |
| ENSG00000196453 | ZNF777 | NS1 | | gene having | |
| | | | | antiviral | |
| | | | | activity | |
| ENSG00000122952 | ZWINT | NS2 | | | |
| ENSG00000070476 | ZXDC | NS1 | | | |
| ENSG00000070476 | ZXDC | NS2 | | | |
| ENSG00000159840 | ZYX | NS2 | | | |
| ENSG00000159840 | ZYX | NS1 | | | |

### REFERENCES

Throughout this application, various references describe the state of the art to which this invention pertains. The disclosures of these references are hereby incorporated by reference into the present disclosure.

1. A method for screening a plurality of substances capable of modulating the replication of an influenza virus in a host cell comprising the step of
identifying a substance that modulates the specific interaction of a host cell protein with a viral protein required for viral replication as depicted in table 1 or
identifying a substance that modulates the specific interaction of a first host cell protein as depicted in table 1 with a second host cell protein present in cellular network of the first host cell protein or
identifying a substance that modulates the expression of a host cell protein as depicted in table 1, or
identifying a substance that modulates the activity of a host cell protein as depicted in table 1.

2. A method for identifying a substance that modulates the specific interaction of a host cell protein of Table 1 with a viral protein required for viral replication, comprising:
in the presence of a test substance, contacting a protein or peptide containing an amino acid sequence corresponding to a binding site of the host cell protein with a protein or peptide having an amino acid sequence corresponding to a binding site of the viral protein, under conditions and for a time sufficient to permit binding and the formation of a complex between the amino acid sequence corresponding to a binding site of the host cell protein and the protein or peptide having an amino acid sequence corresponding to a binding site of the viral protein;
detecting the formation of the complex, wherein an increase or decrease in complex formation compared to an amount of complex formed in the absence of the test substance indicates that the test substance modulates the interaction between the host cell protein and the viral protein; and
selecting test substances which modulate the interaction.

3. The method of claim 2, wherein if said test substance decreases formation of the complex then said test substance is selected as an inhibitor of host cell protein-viral protein interaction.

4. The method of claim 2, wherein if said test substance increases formation of the complex then said test substance is selected as an enhancer of host cell protein-viral protein interaction.
